(19) United States
(12) Patent Application Publication
(10) Pub. No.: US 2021/0379568 A1
(43) Pub. Date: Dec. 9, 2021

(54) METHOD FOR PRODUCING HIGH-EFFICIENCY DEHYDROGENATION CATALYST FOR BRANCHED LIGHT HYDROCARBONS

(71) Applicant: HEESUNG CATALYSTS CORPORATION, Gyeonggi-do (KR)

(72) Inventors: Seung Chul Na, Seoul (KR); Young-san Yoo, Gyeonggi-do (KR); Dong Kun Kang, Gyeonggi-do (KR); Hyun A Choi, Incheon (KR)

(21) Appl. No.: 17/285,503

(22) PCT Filed: Oct. 4, 2019
(86) PCT No.: PCT/KR2019/013026

§ 371 Date: Apr. 15, 2021

(30) Foreign Application Priority Data
Oct. 19, 2018 (KR) 10-2018-0125050

(51) Int. Cl. B01J 23/62; B01J 21/04; B01J 35/00; C07C 5/32

(52) U.S. Cl. CPC B01J 23/626; B01J 21/04; C07C 2523/62; C07C 5/325; C07C 2521/04; B01J 35/008

(57) ABSTRACT

The present disclosure relates to a dehydrogenation catalyst for use in dehydrogenation of a branched light hydrocarbon gas, the catalyst including platinum, tin, and an alkali metal which are carried in a phase-changed carrier, in which platinum and tin form a single complex and are present in an alloy form within a predetermined thickness from the outer surface of the catalyst.

### TECHNICAL FIELD

The present disclosure relates to a method of manufacturing a catalyst for dehydrogenation of branched light hydrocarbons using a stabilized active metal complex, that is, to a catalyst for dehydrogenation of branched C₄-C₇ hydrocarbons. More specifically, the present disclosure relates to a technology of manufacturing a catalyst which contains metal components present in an alloy form within a predetermined thickness on the surface of a carrier and which causes low carbon deposition and has a high conversion rate and selectivity when used in the dehydrogenation of branched hydrocarbons. Particularly, an organic solvent and an organic acid are used when metals are carried, thus manufacturing a catalyst exhibiting high dispersibility and alloying properties.

### BACKGROUND ART

Light olefins are materials used in various commercial applications, such as raw materials for plastics, synthetic rubbers, medicines, and chemical products. Typically, light olefins are extracted as byproducts generated when naphtha derived from crude oil is pyrolyzed, or are extracted from gas produced as a cracking reaction byproduct. Although the global demand for light olefins is increasing every year, the production thereof is limited by conventional production methods. Therefore, the manufacture of light olefins by dehydrogenation using catalysts is continually being researched. Dehydrogenation catalysis is advantageous in that a product having high purity is obtained at high yield compared to a conventional process, and in that the reaction is expected to be easily applicable to manufacturing due to the simple process therefor (Yuling Shan et al., Chem. Eng. J. 278 (2015), p 240). In general, various reactions occur depending on the carbon number of reactants in the dehydrogenation of hydrocarbons, and the main reaction thereof may be expressed as follows.

Branched paraffin (CₙH₂ₙ₊₂)⇔olefin (CₙH₂ₙ)+hydrogen (H₂)

In general, when thermal energy is applied to hydrocarbons, the bond strength between carbon and carbon (240 KJ/mol) is lower than the bond strength between carbon and hydrogen (360 KJ/mol). Accordingly, after the start of the thermodynamic reaction, a carbon-carbon cleavage reaction occurs first, resulting in the generation of byproducts and thus low yield of the product. However, when a suitable catalyst is used, the carbon-carbon cleavage reaction may be minimized, thereby promoting dehydrogenation and thus enabling high yield and selectivity to be secured.

On May 11, 2017, the present applicant filed a method of producing a catalyst having high regeneration efficiency for dehydrogenation of straight-chain light hydrocarbons with the Korean Intellectual Property Office (Patent Application No. 2017-58603), the entire content of which is incorporated herein by reference.

### DISCLOSURE

### Technical Problem

According to the conventional technology, since the alloy form of platinum and tin is manufactured by sequentially carrying platinum and tin, the alloy form of platinum and tin depends only on the probability of contact of the two active materials. In addition to the optimum platinum/tin molar ratio of the target reaction, platinum may be present alone, or another alloy having another platinum/tin molar ratio may be present. In general, optimal results can be achieved only when platinum, which is an active site of dehydrogenation, and tin, which improves the stability of platinum, are present in an alloy form. However, the conventional technology has a problem in that, because platinum or tin is present alone in addition to the platinum-tin alloy, side reactions occur during the reaction. The conventional technology also has the following problems: since a catalyst in which platinum and tin are uniformly distributed in the center of an alumina carrier is used, the catalyst activity is lowered due to carbon (coke) deposited in the alumina during the reaction, and the catalyst is not completely regenerated to the initial state thereof due to the coke that remains therein, and furthermore, is not oxidized, even upon attempting to remove the carbon using a calcination process.

### Technical Solution

According to the present disclosure, in a catalyst for dehydrogenation of branched light paraffinic hydrocarbons, active metals in a carrier are not distributed alone but remain constant in an alloy form, and this alloy is present to a predetermined thickness between the surface of the catalyst and an inner core thereof. In this structure, a high conversion rate and high selectivity are exhibited due to the form of a platinum-tin alloy during the dehydrogenation, and also the amount of carbon that is deposited is generally decreased. Moreover, carbon deposits are not formed due to the absence of an alloy at the center of the catalyst, and carbon deposits are located only at the outer surface of the catalyst where the alloy is distributed. Therefore, an objective of the present disclosure is to provide a catalyst capable of greatly improving catalytic regenerability by completely removing the carbon deposits from the inside of the catalyst upon catalyst regeneration during actual processing and a method of manufacturing the same. The present disclosure is based on the observation that a platinum-tin alloy ratio is not constant when an active metal is directly carried in the conventional technology. Platinum and tin are formed into a composite in an organic solvent, and the composite is carried together with a predetermined amount of organic acid in a carrier so as to be distributed to a predetermined thickness from the surface of an alumina carrier, thereby completing the catalyst.

### Advantageous Effects

According to the present disclosure, a uniform distribution of platinum and tin is obtained in a carrier by using a platinum-tin composite solution, and the conversion rate and selectivity are improved upon dehydrogenation of branched light hydrocarbons by maintaining a platinum-tin alloy ratio constant. A catalyst is manufactured so that a platinum-tin alloy is not present in the carrier. Accordingly, carbon deposition is minimized inside the carrier during a reaction and the amount of carbon that is deposited is generally low.

### DESCRIPTION OF DRAWINGS

FIG. 1 shows the state of the catalyst after reaction showing the characteristics of the present disclosure compared to conventional technology;

FIG. 2 shows a flowchart of steps of the manufacturing process of the present disclosure;

FIG. 3 is a picture of electron probe microanalysis (SPMA) of the catalyst manufactured in Example 1 of the present disclosure and Comparative Example 1; and

FIG. 4 is comparative electron microscope images (video microscopy) showing the catalysts manufactured using the conventional technology and the present disclosure before and after reaction.

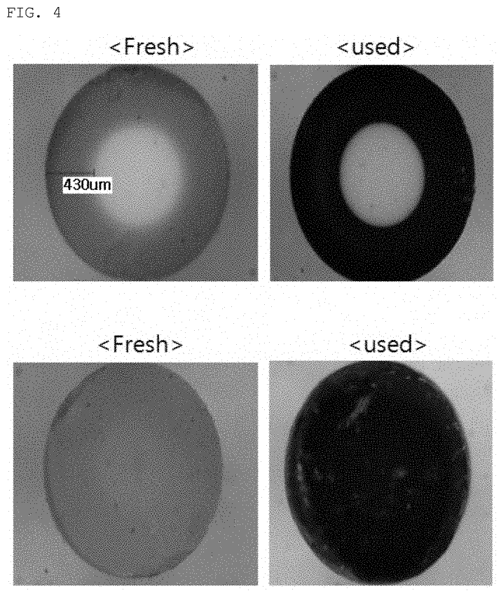

### BEST MODE

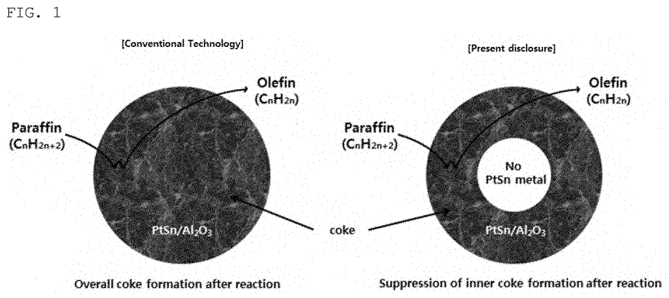

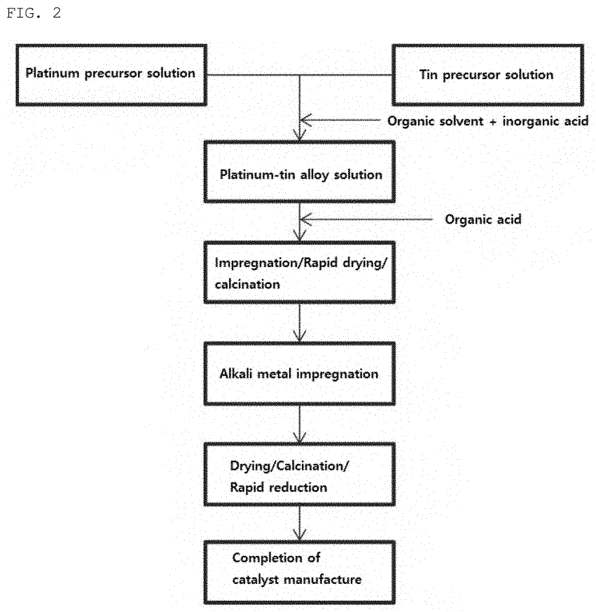

The present disclosure relates to a catalyst for dehydrogenation of branched C₄-C₇ hydrocarbons, and also to a technology of manufacturing a catalyst which contains metal components present in an alloy form in a carrier to a predetermined thickness from the surface of the carrier. The catalyst for dehydrogenation of light hydrocarbons is subjected to a relatively high-temperature reaction compared to heavy hydrocarbons, thus forming a large amount of coke due to thermal decomposition and other side reactions. Therefore, the mass transfer rate depending on the pore size and the pore volume of the carrier may be major factors in the corresponding reaction. Further, when a gas hourly space velocity (GHSV), that is, an addition rate of reactants into a reactor, is high, the amount of carbon deposited in the catalyst increases rapidly. In the catalyst regeneration process that is periodically performed, since the deposited carbon must be easy to remove, it is very important to control the pore distribution in the carrier. Platinum, which is an active metal that directly participates in the reaction, is easily covered with coke when present alone in the carrier. Accordingly, a predetermined amount of an auxiliary metal or alkali metal must always be present around platinum. When the auxiliary metal or alkali metal is present alone in portions of the catalyst rather than being uniformly disposed adjacent to the platinum, adverse results are obtained in terms of both selectivity and durability. Therefore, it was concluded that the use of a catalyst satisfying the above conditions would suppress side reactions in the dehydrogenation, thereby improving the durability and also the conversion rate and selectivity of the catalyst reaction. Surprisingly, the present inventors found that when the active metals are not distributed alone in the carrier but are present in an alloy form to a predetermined thickness from the surface of the catalyst to the inside thereof in the case of the catalyst for dehydrogenation of branched light paraffinic hydrocarbons, it is possible to manufacture a catalyst capable of greatly increasing the conversion rate of branched paraffins, particularly isobutane, olefin selectivity, and durability. The present disclosure provides a method of manufacturing a catalyst capable of controlling the distribution of an active metal to a predetermined thickness from the surface of the catalyst by carrying an alloy-type active metal formed using an organic solvent together with a predetermined amount of an organic acid and/or an inorganic acid. FIG. 1 shows the core technology of the present disclosure for comparison with a conventional technology, and FIG. 2 shows a flowchart of a method of manufacturing a catalyst, which comprehensively explains the method of the present disclosure.

1) Step of Manufacturing Stabilized Platinum-Tin Composite Solution

The composite solution of platinum and tin facilitates precipitation of platinum in air due to the high reducibility of tin. Therefore, selection of a solvent is very important in the manufacture of the composite solution. When water is used as the solvent, since tin reduces platinum, a platinum-tin precursor solution remains very unstable, and eventually platinum particles are precipitated, which makes the solution unusable as a precursor. Therefore, the present inventors manufactured a precursor solution that is maintained in a stable state over time using a solvent that does not reduce tin. First, the precursors of platinum and tin were added to the organic solvent in the state of being mixed with each other so that the platinum-tin composite was not decomposed, and hydrochloric acid was added to manufacture an acidic solution. Then, an organic acid was added in order to increase the speed of penetration into the inside of the carrier. In the case of the organic solvent, one or two among water, methanol, ethanol, butanol, acetone, ethyl acetate, acetonitrile, ethylene glycol, triethylene glycol, glycol ether, glycerol, sorbitol, xylitol, dialkyl ether, and tetrahydrofuran may be sequentially used, or may be used in the form of a mixed solution. In the case of the organic acid, one or two among formic acid, acetic acid, glycolic acid, glyoxylic acid, oxalic acid, propionic acid, and butyric acid, among carboxylic acids, may be mainly used as a mixed solution. During the manufacture of the platinum-tin composite solution, the solution is aged in an inert gas atmosphere, thus suppressing decomposition by oxygen and achieving stabilization. Nitrogen, argon, and helium may be used as the inert gas, with nitrogen gas being preferably used.

2) Step of Manufacturing Catalyst Using Stabilized Platinum-Tin Composite Solution and Alkali Metal

In order to increase the pore size and the pore volume, the carrier is heat-treated in a calcination furnace at 1000 to 1050° C. for 1 to 5 hours, whereby gamma alumina is phase-changed to theta alumina for use. The heat treatment temperature is closely related to the crystal phase and the pore structure of the carrier. If the heat treatment temperature is lower than 1000° C., the crystal phase of alumina is in a state in which gamma and theta are mixed with each other, and the pore size of the carrier is small, and thus the diffusion rate of reactants in the carrier may be lowered. On the other hand, if the heat treatment temperature is higher than 1050° C., the crystal phase of alumina is in a state in which theta and alpha phases are mixed with each other, and thus the pore size is favorable to the reaction, but the dispersibility of the active metals distributed on the alpha alumina is lowered during a process of carrying the active metals. In the process of carrying the active metals, a platinum-tin composite solution is manufactured in an amount equivalent to the total pore volume of the carrier, and is impregnated into the carrier using a spraying process. After the impregnation, an aging process is performed for a predetermined period of time in order to control the penetration depth of platinum and tin into alumina using an organic acid. After the aging process, a rapid drying process is performed while fluidizing the catalyst in an atmosphere heated to 150 to 250° C., thus removing most of the organic solvent remaining in the catalyst. Water remaining in the catalyst is completely removed via a drying process at 100 to 150° C. for 24 hours. The reason for performing rapid drying is to prevent the platinum-tin composite solution from diffusing into the carrier together with an inorganic or organic acid solvent over time when the platinum-tin composite solution is carried in the alumina carrier. Rapid drying at lower than 150° C. is insufficient to fix the metals, whereas rapid drying at higher than 250° C. may cause aggregation of metal particles due to the decomposition of an organic solvent. After drying, an organic material is removed under a nitrogen atmosphere at 250 to 400° C., followed by a calcination process in an ambient atmosphere at 400 to 700° C. If the heat treatment is performed at lower than 400° C., the carried metal may not be converted into metal oxide species, whereas if the heat treatment is performed at higher than 700° C., an intermetallic aggregation phenomenon occurs, and the catalyst activity is not high considering the amount of the catalyst. After calcination, a step for carrying alkali metal is performed in order to suppress catalyst side reactions. First, potassium is carried in the internal pores of the carrier using the same spraying process as in the case of the above-mentioned platinum-tin composite solution, and a drying process at 100 to 150° C. for 24 hours and a calcination process in an ambient atmosphere at a temperature in the range of 400 to 700° C. are performed. Finally, after the calcination, a reduction process is performed using a hydrogen/nitrogen mixed gas (in a composition range of 4%/96% to 100%/0%) at a temperature in the range of 400 to 600° C., thus obtaining a final catalyst. When a reduction temperature is lower than 400° C. during the reduction process, the metal oxide species may not be completely reduced, and two or more kinds of metal particles may be present as individual metals rather than in an alloy form. Further, when the reduction temperature is higher than 600° C., aggregation and sintering occur between two or more kinds of metal particles, and as a result, the catalyst activity may be lowered as the number of active sites decreases. The reduction is performed using a rapid high-temperature reduction method in which a nitrogen atmosphere is maintained until a predetermined temperature is reached and hydrogen gas is injected to perform the reduction when the predetermined temperature is reached, instead of a temperature-raising reduction method in which reduction is performed using hydrogen gas from a temperature-raising step. When the reduction is performed using the temperature-raising reduction method, there is a problem in that, since the reduction temperatures of platinum and tin are different from each other, they are present in the form of individual metals in the catalyst after the reduction, so the role of tin cannot be maximized in terms of coke suppression and durability.

The performance of the catalyst manufactured as described above is evaluated as follows. Conversion of branched light paraffin hydrocarbons into olefins may be performed using a gas-phase reaction under conditions of 500 to 680° C., preferably 570° C., 0 to 2 atm, preferably 1.5 atm, and a branched paraffin hydrocarbon GHSV (gas hourly space velocity) of 500 to 10000 h⁻¹, preferably 2000 to 8000 h⁻¹, by diluting hydrocarbons having 4 to 7 carbon atoms, preferably 4 to 5 carbon atoms, including isoparaffin, with hydrogen using the dehydrogenation catalyst according to the present disclosure. The reactor for producing olefins using the dehydrogenation is not particularly limited, but a fixed-bed catalytic reactor in which the reactor is filled with a catalyst may be used. Further, since dehydrogenation is an endothermic reaction, it is important that the catalyst reactor always be maintained under adiabatic conditions. For the dehydrogenation process of the present disclosure, it is important to perform the reaction while maintaining a reaction temperature, a pressure, and a liquid hourly space velocity, which are reaction conditions, within suitable ranges. When the reaction temperature is low, the reaction does not proceed. When the reaction temperature is very high, the reaction pressure is increased in proportion thereto, and side reactions such as coke formation and cracking reactions occur.

### Example 1: Manufacture of Catalyst Using Simultaneous Platinum-Tin Impregnation Process

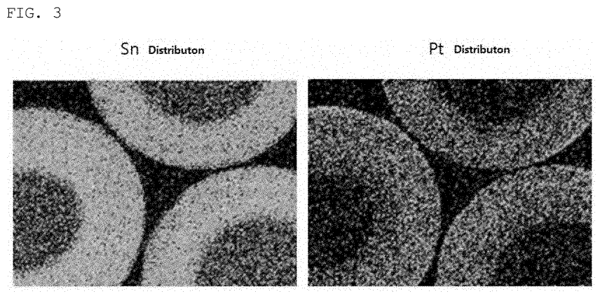

With respect to the carrier used in Example 1, a gamma alumina carrier (Manufacturer: BASF in Germany, specific surface area: 210 m²/g, pore volume: 0.7 cm³/g, average pore size: 8.5 nm) was calcinated at 1020° C. for 5 hours so as to be phase-changed into theta alumina, and the resultant theta alumina carrier was used. The phase-changed theta alumina has physical properties including a specific surface area of 92 m²/g, a pore volume of 0.41 cm³/g, and an average pore size of 12 nm. Chloroplatinic acid (H₂PtCl₆) was used as a platinum precursor and tin chloride (SnCl₂) was used as a tin precursor. The solvent that was used was prepared using 97 wt % of ethanol and 3 wt % of hydrochloric acid. The tin chloride and the platinum precursor were dissolved in 3 wt % of hydrochloric acid and then mixed with 97 wt % of ethanol. In addition, glyoxylic acid was mixed therewith in an amount equivalent to 3 wt % of the total amount of the solvent in order to realize flowability of a platinum-tin alloy solution in the carrier. Thereafter, the theta alumina carrier having undergone phase change was impregnated with the manufactured platinum-tin composite solution using a spraying process. After the impregnation, an aging process was performed at room temperature for about 30 minutes. Thereafter, drying was performed at 120° C. for 12 hours to thus completely remove the organic solvent and moisture from the catalyst, followed by heat treatment at 550° C. for 3 hours in an ambient atmosphere, thereby fixing the active metal. Next, potassium nitrate (KNO₃) was dissolved in less than 1 wt % of nitric acid (HNO₃) and 99 wt % of deionized water to afford a potassium solution, which was then carried into the internal pores of alumina containing platinum and tin using a spraying process. The composition in which metal was carried was dried in an ambient atmosphere at 120° C. for 12 hours or more, thus completely removing moisture from the catalyst, and was then heat-treated at 550° C., thus manufacturing a metal-carried catalyst. The catalyst reduction process was performed in a stepwise manner, in which the temperature was raised to 500° C. in an ambient atmosphere, purging with nitrogen was performed for about 5 to 10 minutes, and hydrogen gas was then allowed to flow, thereby manufacturing the reduced catalyst. The catalyst manufactured in Example 1 contained 0.4 wt % of platinum, 0.17 wt % of tin, and 8.8 wt % of potassium, and the state of active metals, determined through electron probe microanalysis (EPMA), is shown in FIG. 3. As a result, it was confirmed that platinum and tin were uniformly distributed in a form resembling an egg shell in the catalyst.

### Comparative Example 1: Manufacture of Catalyst Using Sequential Impregnation of Platinum and Tin

With respect to the carrier used in Comparative Example 1, as in Example 1, gamma alumina was calcinated at 1050° C. for 2 hours so as to be phase-changed into theta alumina, and the resultant theta alumina was used. As a tin precursor, tin chloride (SnCl₂) was diluted in deionized water and in an inorganic acid in an amount equivalent to 5 wt % of the total amount of the solvent and carried in the pores in the alumina using a spraying process, followed by drying at 120° C. for 12 hours or more to completely remove moisture and then heat treatment at 650° C. in an ambient atmosphere, thereby fixing the active metal. Chloroplatinic acid (H₂PtCl₆) as a platinum precursor was diluted in deionized water in an amount equivalent to the total pore volume of the carrier and in an inorganic acid in an amount equivalent to 5 wt % of the total amount of the solvent, and was then impregnated in a carrier using a spraying process. After drying at 120° C. for 12 hours and then heat treatment at 550° C. for 3 hours in an ambient atmosphere, the active metal was fixed. Thereafter, potassium was carried in the pores in alumina containing platinum and tin in the same manner as in Example 1. The catalyst thus manufactured contained 0.4 wt % of platinum, 0.17 wt % of tin, and 8.8 wt % of potassium.

### Experimental Example 1: Evaluation of Catalyst Performance

Dehydrogenation was performed in order to measure the catalyst activity, and a reactor was evaluated using a fixed-bed reaction system. 1 ml of the catalyst was charged in a tubular reactor, and the temperature was raised while hydrogen gas was allowed to flow at a constant rate of 12 cc/min, after which the catalyst was maintained at that temperature for 20 minutes. Subsequently, a mixed gas of hydrogen gas and isobutane gas, which were the raw materials used in the reaction, mixed at a ratio of 0.4, was continuously supplied to the reactor, and a gas hourly space velocity was constantly fixed at 8100 h⁻¹. Further, hydrogen sulfide gas in an amount equivalent to 100 ppm of the total amount of reactants was further injected in order to suppress side reactions occurring during the catalytic reaction. The materials generated at individual temperatures were introduced to a GC (gas chromatograph) through an injection line wrapped with hot wires, and quantitative analysis was performed using a FID (flame ionization detector). The above experiment was performed at temperatures of 590° C. and 615° C. The conversion rate of isobutane and the isobutylene selectivity for the product were calculated as shown below, and the activities of the catalysts were compared with each other using the yield of propylene obtained thereby.

Conversion rate of isobutane (%)=[number of moles of isobutane before reaction−number of moles of isobutane after reaction]/[number of moles of isobutane]×100

Selectivity of isobutylene (%)=[number of moles of isobutylene in product]/[number of moles of product]×100

Yield of isobutylene (%)=[conversion rate of isobutane]×[selectivity of isobutylene]/100

The results of activity test of the catalysts manufactured in Example 1 and Comparative Example 1 and the amount of coke that was deposited are shown in Table 1 below.

TABLE 1

| | | Isobutene | Isobutylene | Isobutylene | Coke |
|---|---|---|---|---|---|
| Temperature | | conversion rate | selectivity | yield | deposition |
| (° C.) | Classification | (%) | (%) | (%) | (%) |
| 590 | Example 1 | 51.4 | 88.5 | 45.5 | 0.69 |
| | Comparative | 46.6 | 87.6 | 40.8 | 1.05 |
| | Example 1 | | | | |
| 615 | Example 1 | 62.2 | 83.4 | 51.9 | 1.23 |
| | Comparative | 56.2 | 82.4 | 46.3 | 1.91 |
| | Example 1 | | | | |

As is apparent from the results of Table 1, it can be seen that, when the reaction temperature was raised from 590° C. to 615° C., the conversion rate increased, the selectivity decreased, and the coke deposition increased. This phenomenon is deemed to appear because thermal cracking increased at the high temperature due to the raised activation temperature. The catalyst of Example 1, in which platinum and tin were impregnated to a predetermined thickness in the carrier in an alloy form, exhibited the best activity in terms of conversion rate and selectivity at both reaction temperatures of 590° C. and 615° C., and the coke deposition was the lowest. In Example 1, platinum and tin were distributed to the same thickness of 500 μm beneath the surface of the carrier and were present in the form of a platinum-tin alloy, so side reactions due to the use of platinum or tin alone were suppressed, thereby exhibiting a high conversion rate and selectivity. However, the catalyst of Comparative Example 1 was manufactured using the sequential impregnation process, and exhibited low conversion rate and selectivity compared to the simultaneous impregnation process. This is deemed to be because platinum and tin were not impregnated together but were impregnated sequentially and thus the platinum-tin alloy ratio was lower than that of Example 1, and coke was also confirmed to be generated in a large amount due to the use of platinum alone.

1. A dehydrogenation catalyst for use in dehydrogenation of a branched light hydrocarbon gas, comprising:
platinum, tin, and an alkali metal, which are carried in a phase-changed carrier,
wherein the platinum and the tin form a single complex and are present in an alloy form within a predetermined thickness from an outer surface of the catalyst.

2. The dehydrogenation catalyst of claim 1, wherein a molar ratio of the platinum to the tin in the complex of platinum and tin is 0.5-3.0.

3. The dehydrogenation catalyst of claim 1, wherein the platinum and the tin are manufactured so that distances from a surface of the carrier to a center thereof are identical to each other.

4. The dehydrogenation catalyst of claim 1, wherein the catalyst is manufactured so that the single complex is distributed to a thickness of 200 to 600 μm from the outer surface of the catalyst.

5. The dehydrogenation catalyst of claim 1, wherein the carrier is selected from the group consisting of alumina, silica, zeolite, and a composite component thereof.

6. A method of dehydrogenating a branched hydrocarbon, comprising bringing a branched hydrocarbon gas into contact with the catalyst of claim 1 under dehydrogenation conditions.

7. The method of claim 6, wherein the hydrocarbon gas comprises a hydrocarbon gas that has 4 to 7 carbon atoms and enables dehydrogenation.
